(19) United States
(12) Patent Application Publication
(10) Pub. No.: US 2026/0334915 A1
(43) Pub. Date: Oct. 29, 2026

(54) WEARABLE DEVICES HAVING 4D ULTRASOUND IMAGING AND RELATED TECHNIQUES

(71) Applicant: Massachusetts Institute of Technology, Cambridge, MA (US)

(72) Inventor: Colin R. MARCUS, Cambridge, MA (US)

(73) Assignee: Massachusetts Institute of Technology, Cambridge, MA (US)

(21) Appl. No.: 19/656,473

(22) Filed: Apr. 23, 2026

Related U.S. Application Data
(60) Provisional application No. 63/793,123, filed on Apr. 23, 2025.

(51) Int. Cl. A61B 8/00; G01S 7/534; G01S 15/89

(52) U.S. Cl. CPC A61B 8/4488; A61B 8/4209; G01S 7/534; G01S 15/895; G01S 15/8993

(57) ABSTRACT

An ultrasound imaging system can include a two-dimensional transducer array having a plurality of transmit elements and a plurality of receive elements, a transmit circuit for driving the transmit elements with an excitation signal, a receive circuit for receiving an analog signal from each of the receive elements and producing a digital signal for each of the receive elements, and a processor for constructing a volumetric image from the digital signals. The transmit circuit can generate and/or otherwise provide the excitation signal at a swept-frequency, which can vary over time. Each of the transmit elements and each of the receive elements can occupy distinct, non-overlapping positions. Each transmit element can emit acoustic energy into a medium, such as in response to the excitation signal. Each receive element can provide the analog signal in response to acoustic energy received from the medium.

### CROSS-REFERENCE TO RELATED APPLICATIONS

This application claims benefit of U.S. Provisional Patent Application No. 63/793,123, filed Apr. 23, 2025, the entirety of which is incorporated herein by specific reference.

### STATEMENT REGARDING FEDERALLY SPONSORED RESEARCH

This Invention was made with government support under 2044688 awarded by the National Science Foundation. The government has certain rights in the invention.

### BACKGROUND

Wearable ultrasound devices face many challenges. Most probes image in a narrow (typically <3 mm) two-dimensional (2D) plane, which is unlikely to intersect the region of interest without careful manual manipulation. Even with careful placement, imperceptible tilting of a transducer can cause a large displacement of the imaging plane. Proposed solutions have included adding additional arrays or overlaying multiple arrays to take multiple brightness mode (B-mode) slices at different angles; however, these are insufficient to address the problem. Attempting to intersect a randomly located target within a volume using thin planar slices is simply not reliable enough to form the basis of an operator-independent medical technology.

One solution would be wide-angle volumetric imaging; however existing three-dimensional video (4D) imaging architectures present critical tradeoffs that preclude their use in a wearable format. Currently, three main classes of architectures exist: full matrix, row-column, and sparse arrays. Full matrix arrays implement a full 2D matrix of elements, offering good performance and a wide field of view (FOV), but also leading to very high element counts scaling with current state-of-the-art commercial systems supporting up to 256×256 or 65,536 elements. Such arrays are challenging and expensive to fabricate, and require bulky and power intensive electronics to interface with all the elements.

Row-column addressed arrays reduce the wiring complexity by operating the transducer as a one dimensional (1D) linear array on one axis for transmit (TX), and switching to an orthogonal axis for receive (RX). This reduces the number of signals to a linear solution from a quadratic solution (e.g., in Big O notation, O(4N) versus O(N²)). However, row-column arrays can only form images in the region where the orthogonal transmit and receive imaging planes overlap. Thus, the imaging region is confined to the footprint of the array itself, forming a “pencil beam” that actually poses even harsher requirements for precise positioning and tilting than the B-mode images.

Finally, sparse arrays are essentially full matrix arrays with most of the elements removed. As such, they are capable of wide-angle imaging. A number of designs have been proposed over the years with random, optimized-random, and other geometries. However, as the number of elements is reduced from a quadratic solution (e.g., O(N²)) towards a linear solution (e.g., O(4N)), for a given size, the arrays increasingly suffer from image artifacts, elevated sidelobes, poor contrast performance, and low imaging depth due to the reduced active area of the array.

### SUMMARY

Applicant has created new and useful devices, systems and methods for a novel ultrasound imaging system that can include a convolutional optimally distributed array (CODA) geometry that drastically reduces the number of elements and power consumption, a chirped data acquisition (cDAQ) architecture that boosts signal to noise ratio (SNR), and an associated signal processing methodology. A CODA according to the disclosure can include a box-shaped array with separated transmit and receive subarrays, with an analog front end which uses continuous wave (CW) transmit chirp signals and low frequency receiver circuits to increase the SNR while simultaneously consuming less power than previous ultrasound systems.

An ultrasound imaging system according to the disclosure can include a transducer array having one or more transmit elements and one or more receive elements, a transmit circuit for driving the transmit elements with an excitation signal, a receive circuit for receiving an analog signal from each of the receive elements and producing a digital signal for each of the receive elements, a processor for constructing a volumetric image from the digital signals, or any combination thereof. The transmit circuit can generate and/or otherwise provide the excitation signal at a swept-frequency. The swept-frequency can vary over time, such as across a predetermined bandwidth, and/or have a center frequency. The transducer array can be two-dimensional and include a plurality of transmit elements and/or a plurality of receive elements. Each of the transmit elements and each of the receive elements can occupy distinct, non-overlapping positions. Each transmit element can emit acoustic energy into a medium, such as in response to the excitation signal. Each receive element can provide the analog signal in response to acoustic energy received from the medium. In at least one embodiment, the transmit elements can emit acoustic energy into the medium continuously or repeatedly and/or the receive elements can receive acoustic energy from the medium and/or provide the analog signal continuously or repeatedly, such as while the transmit elements are emitting acoustic energy into the medium.

The transmit elements and the receive elements can be disposed on a substrate, such as to define a virtual aperture between them. The substrate can be thinner between adjacent elements, such as between a transmit element and an adjacent receive element or subarrays thereof, which can reduce parasitic coupling between the transmit elements and the receive elements. The virtual aperture can be substantially fully sampled and/or substantially uniformly weighted. The transmit elements and the receive elements can be disposed between the substrate and a conductive backing layer, which can provide shielding, decoupling, otherwise prevent electro-magnetic interference, or any combination thereof.

The transmit elements and the receive elements can be arranged along sides of a polygonal perimeter region, such as a square or rectangle. For example, the transmit elements can be arranged along a first subset of sides of the polygonal perimeter region and/or the receive elements can be arranged along a second subset of sides of the polygonal perimeter region. The first subset of sides and the second subset of sides can be mutually exclusive, such that no side of the polygonal perimeter region contains both transmit and receive elements. The transmit elements can be arranged orthogonally to the receive element and/or the transmit elements and the receive elements can be arranged along opposing sides.

For example, in at least one embodiment, a plurality of transmit elements can be arranged on a substrate along a first side of a polygonal perimeter region to form a first subarray, a plurality of receive elements can be arranged on the substrate along a second side of the polygonal perimeter region to form a second subarray, another plurality of transmit elements can be arranged on the substrate along a third side of a polygonal perimeter region to form a third subarray, another plurality of receive elements can be arranged on the substrate along a fourth side of the polygonal perimeter region to form a fourth subarray, or any combination thereof. The first side and the third side can be opposing and/or the second side and the fourth side can be opposing, such as where the polygonal perimeter region is rectangular. In at least one embodiment, no side of the polygonal perimeter region contains both transmit elements and receive elements.

The transmit elements in the first subarray and/or the transmit elements in the third subarray can be spaced at a first pitch. The receive elements in the second subarray and/or the receive elements in the fourth subarray can be spaced at a second pitch. The first subarray and/or the third subarray can be offset from the second subarray and/or fourth subarray by approximately one half of the second pitch. The second subarray and/or the fourth subarray can be offset from the first subarray and/or third subarray by approximately one half of the first pitch.

The transmit elements and/or the receive elements can be generally linearly aligned in the various subarrays. In at least one embodiment, not all elements need to be perfectly aligned in the various subarrays. For example, the transmit elements at each end of the first subarray can be disposed further from the third subarray than the other transmit elements in the first subarray, the receive elements at each end of the second subarray can be disposed further from the fourth subarray than the other receive elements in the second subarray, the transmit elements at each end of the third subarray can be disposed further from the first subarray than the other transmit elements in the third subarray, the receive elements at each end of the fourth subarray can be disposed further from the second subarray than the other receive elements in the fourth subarray, or any combination thereof.

The transmit circuit, the receive circuit, the processor, or any combination thereof can be embodied in a single integrated circuit. The transducer array can fully or partially surround the integrated circuit. Electrical connections between the integrated circuit and the transducer array can extend radially outwards from the integrated circuit.

The receive circuit can include one or more delta-sigma analog-to-digital converters that can sample the analog signals from the receive elements, such as at a sampling rate based at least in part on the swept-frequency. In at least one embodiment, the receive circuit can include one or more analog frequency downconverters and/or one or more analog-to-digital converters. The analog frequency downconverter can produce an intermediate-frequency signal for each of the receive elements, such as by mixing the analog signal from respective receive elements with a reference signal. The reference signal can be based at least in part on the swept-frequency of the excitation signal. The intermediate-frequency signal for each of the receive elements can include an echo time delay, which can be encoded as a proportional frequency. The analog-to-digital converter can produce the digital signal for each of the receive elements, such as by sampling the intermediate-frequency signal for each of the receive elements at a sampling rate. The sampling rate can be less than the center frequency of the swept-frequency, such as less than half the center frequency or an order of magnitude (or more) less than the center frequency.

An ultrasound transducer system according to the disclosure can include a plurality of transmit elements arranged along at least a first side of a polygonal perimeter region for emitting acoustic energy into a medium in response to an excitation signal and/or a plurality of receive transducer elements arranged along at least a second side of the polygonal perimeter region for providing an analog signal in response to acoustic energy received from the medium. The first side and the second side can be mutually exclusive, such as so that no side of the polygonal perimeter region contains both transmit elements and receive elements. A two-dimensional spatial convolution of the transmit elements with the receive elements can define a fully sampled, unity-valued two-dimensional array having no unsampled spatial positions and no redundantly sampled spatial positions within its extent.

The polygonal perimeter region can have three or more sides. In at least one embodiment, the polygonal perimeter region can have an even number of sides. The polygonal perimeter region can be rectangular having four sides, such as the first side, the second side, a third side, and a fourth side. The first side and the third side can be opposing and/or the second side and the fourth side can be opposing, such as with the transmit elements arranged along the first side and the third side and/or the receive elements arranged along the second side and the fourth side.

The transmit elements on the first side can be spaced at a first pitch to form a first subarray, the receive elements on the second side can be spaced at a second pitch to form a second subarray, the transmit elements on the third side can be spaced at the first pitch to form a third subarray, the receive elements on the fourth side can be spaced at the second pitch to form a fourth subarray, or any combination thereof. The first subarray and/or the third subarray can be offset from the second subarray and/or fourth subarray by approximately one half of the second pitch. The second subarray and/or the fourth subarray can be offset from the first subarray and/or third subarray by approximately one half of the first pitch.

The transmit elements at each end of the first subarray can be disposed further from the third subarray than other transmit elements in the first subarray, the receive elements at each end of the second subarray can be disposed further from the fourth subarray than other receive elements in the second subarray, the transmit elements at each end of the third subarray can be disposed further from the first subarray than other transmit elements in the third subarray, the receive elements at each end of the fourth subarray can be disposed further from the second subarray than other receive elements in the fourth subarray, or any combination thereof.

The transmit elements and/or the receive elements can be disposed on a substrate. The substrate can be thinner between adjacent subarrays. The transmit elements and/or the receive elements can be disposed between the substrate and a conductive backing layer.

### BRIEF DESCRIPTION OF THE DRAWINGS

This application contains at least one drawing executed in color. Copies of this patent or patent application publication with color drawing(s) will be provided by the Office upon request and payment of the necessary fee.

The manner and process of making and using the disclosed embodiments may be appreciated by reference to the figures of the accompanying drawings. It should be appreciated that the components and structures illustrated in the figures are not necessarily to scale, emphasis instead being placed upon illustrating the principals of the concepts described herein. Like reference numerals designate corresponding parts throughout the different views. Furthermore, embodiments are illustrated by way of example and not limitation in the figures, in which:

FIG. 1 is a simplified block diagram of one of many embodiments of an ultrasound imaging system according to the disclosure.

FIG. 2 is a simplified block diagram of another one of many embodiments of an ultrasound imaging system according to the disclosure.

FIG. 3 is a simplified block diagram of a portion one of many embodiments of an ultrasound imaging system according to the disclosure.

FIG. 4 is a simplified block diagram of a portion one of many embodiments of an ultrasound imaging system according to the disclosure.

FIG. 5 is a simplified block diagram of one of many embodiments of a transducer array according to the disclosure.

FIG. 6 is a representation of one of many embodiments of a transducer array according to the disclosure, shown with little to no offset between subarrays.

FIG. 7 is a representation of one of many embodiments of a transducer array according to the disclosure, shown with offset between subarrays.

FIG. 8 is a simplified block diagram of another one of many embodiments of a transducer array according to the disclosure, shown with end elements offset within subarrays.

FIG. 9 is a representation of one of many embodiments of a transducer array according to the disclosure, shown with end elements offset within subarrays.

FIG. 10 is a simplified perspective view of one of many embodiments of a transducer array according to the disclosure.

FIG. 11 is a simplified block diagram of select components of one of many embodiments of an ultrasound imaging system according to the disclosure.

FIG. 12 is a perspective view of one of many embodiments of an ultrasound imaging system according to the disclosure.

FIG. 13 is a perspective exploded view of select components of one of many embodiments of an ultrasound imaging system according to the disclosure.

FIG. 14 is a graph of one of many embodiments of an excitation signal and one of many embodiments of a received analog signal in the frequency domain.

FIG. 15 is a graph of the received analog signal of FIG. 14, and other signals, shown in the time domain.

FIG. 16 is a graph of one of many embodiments of an excitation signal in the time domain.

### DETAILED DESCRIPTION

Before describing the concepts, devices, systems and techniques sought to be protected herein, some introductory concepts are explained. The concepts described herein are directed towards wearable devices having four-dimensional (4D) ultrasound imaging techniques. As devices are wearable, it is desirable to provide an ultrasound device which reduces (and ideally, minimizes) the amount of heat generated by the ultrasound device compared with the amount of heat generated by conventional ultrasound devices. To accomplish this, the inventors have recognized and appreciated concepts to reduce (and ideally minimize) the hardware requirements and amount of hardware used to provide wearable devices capable of providing 4D ultrasound images. As will be described in detail hereinbelow, a wearable ultrasound device in accordance with the concepts described herein utilizes a sparse transducer array, a swept frequency excitation signal (e.g., a chirp transmit signal), analog down conversion and low frequency sampling to provide volumetric imaging. While each individual part of the system suffers from its own unique set of weaknesses, the weaknesses of each individual component of the system are compensated using strengths of other individual component of the system.

An ultrasound imaging system 100 according to the disclosure can include one or more transducer arrays 200 having one or more transmit elements 202 and one or more receive elements 204, one or more transmit circuits 300 for driving the transmit elements 202 with one or more excitation signals 302. The transmit circuit 300 can generate and/or otherwise provide the excitation signal 302 at a swept-frequency (e.g. an up-chirp signal in which the signal frequency increases from a first frequency to a second frequency between a start time and an end time or a down-chirp signal in which the signal frequency decreases from a first frequency to a second frequency between a start time and an end time). The swept-frequency can vary over time, such as across a predetermined bandwidth, and/or have a center frequency. Each transmit element 202 can emit acoustic energy into a medium, such as in response to the excitation signal 302.

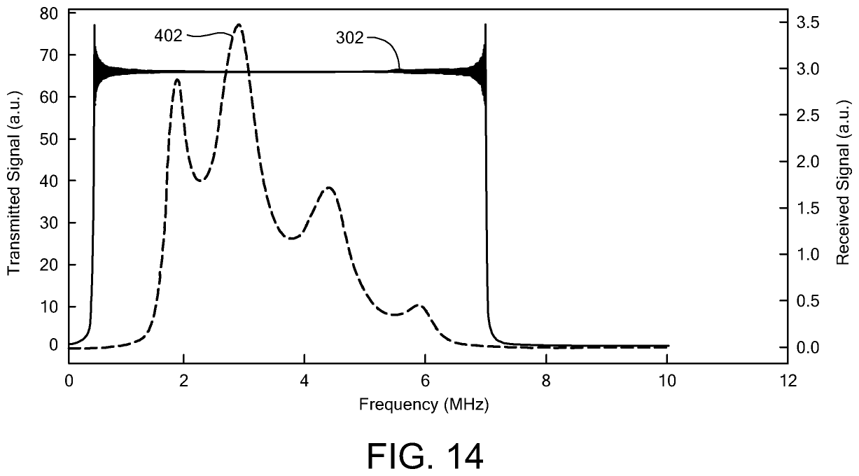

The excitation signal 302 can be a sine wave, a square wave, a triangle wave, another waveform, or any combination thereof. The excitation signal 302 can be of constant power, such as shown in FIG. 14. In exemplary embodiments, the excitation signal 302 can have a variable power envelope, which can compensate for the frequency response of the transmit elements 202 and/or the receive elements 204. The predetermined bandwidth and/or the center frequency of the excitation signal 302 can be determined according to a desired imaging depth, desired imaging resolution, characteristics of the medium, characteristics of the object or target of interest within the medium, other factors, or any combination thereof.

Ultrasound imaging system 100 further includes one or more receive circuits 400 for receiving one or more analog signals 402 from the receive elements 204 and producing one or more digital signals for each of the receive elements 204 as is generally known. Each receive element 204 can provide the analog signal 402 in response to acoustic energy received from the medium. The medium can include living tissue, such as where the ultrasound imaging system 100 is used to detect abnormalities and/or otherwise diagnose one or more conditions.

Ultrasound imaging system 100 further includes one or more processors 500 for constructing one or more volumetric images from the digital signals, or any combination thereof.

The transducer array 200 can be two-dimensional and include a plurality of transmit elements 202 and/or a plurality of receive elements 204. For example, in at least one embodiment, the transducer array 200 can include 64 transmit elements 202 and 64 receive elements 204, but may include other numbers of transmit elements 202 and/or other numbers of receive elements 204. Each of the transmit elements 202 and/or each of the receive elements 204 can occupy distinct, non-overlapping positions. In at least one embodiment, a two-dimensional spatial convolution of the transmit elements 202 and the receive elements 204 can define a fully sampled, unity-valued two-dimensional array 200 having no unsampled spatial positions and no redundantly sampled spatial positions within its extent.

The transmit elements 202 and the receive elements 204 can be disposed on a substrate 206, such as to define a virtual aperture between them. The virtual aperture can be substantially fully sampled and/or substantially uniformly weighted. The substrate 206 can be thinner between adjacent elements 202, 204, such as between a transmit element 202 and an adjacent receive element 204 or subarrays thereof, which can reduce parasitic coupling between the transmit elements 202 and the receive elements 204, among other things.

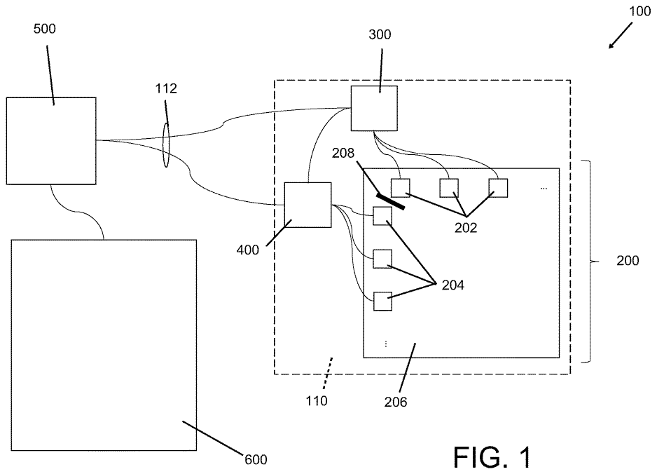

For example, as shown in FIG. 1, a portion 208 of the substrate 206 can be removed, such as through laser etching or micro-machining, between adjacent elements 202, 204 of neighboring subarrays.

As shown in FIG. 4, for example, the transmit elements 202 and the receive elements 204 can be disposed between the substrate 206 and a conductive backing layer 210, which can provide shielding, decoupling, or otherwise prevent electro-magnetic interference with the elements 202, 204 and/or signals 302, 402. The conductive backing layer 210 can abut and/or be offset from the transmit elements 202, the receive elements 204, the substrate 206 or any combination thereof. The conductive backing layer 210 can have a high acoustic impedance, such as to match the acoustic impedance of the transmit elements 202 and the receive elements 204, and/or a high acoustic attenuation, such as to absorb the acoustic energy received from the medium. For example, by matching the acoustic impedance of the transmit elements 202 and the receive elements 204, reflection of the acoustic energy received from the medium can be reduced and/or eliminated.

In at least one embodiment, the conductive backing layer 210 can be or include a tungsten powder suspended in an epoxy and/or a viscoelastic material, such as urethane or another rubber. The conductive backing layer 210 can be applied as a paste or liquid over the transmit elements 202, the receive elements 204, the substrate 206, interconnections, other components, or any combination thereof. For example, a polymer or other electrically insulating material can be applied, such as through physical vapor deposition (PVD), chemical vapor deposition (CVD), plasma polymerization, or sputtering, and then the conductive backing layer 210 can be applied to cover the transmit elements 202, the receive elements 204, the substrate 206, interconnections, other components, or any combination thereof. As another example, the substrate 206, with the transmit elements 202, the receive elements 204, the substrate 206, interconnections, other components, or any combination thereof can be dip coated, spin coated, or spray coated with a low viscosity epoxy/polymer such as Epotek 301. In at least one embodiment, the polymer or other insulating material can be less than 10 micrometers thick, such as to prevent interfering with the acoustic properties.

In at least one embodiment, a non-conductive backing layer can be similar to the conductive backing layer 210 and include an additional insulating ceramic powder such as zirconium oxide, which can break up/interrupt chains of tungsten particles and/or otherwise prevent the non-conductive backing layer from becoming conductive once cured. For example, where electro-magnetic interference is less of a concern, the non-conductive backing layer can have a high acoustic impedance, such as to match the acoustic impedance of the transmit elements 202 and the receive elements 204, and/or a high acoustic attenuation, such as to absorb the acoustic energy received from the medium.

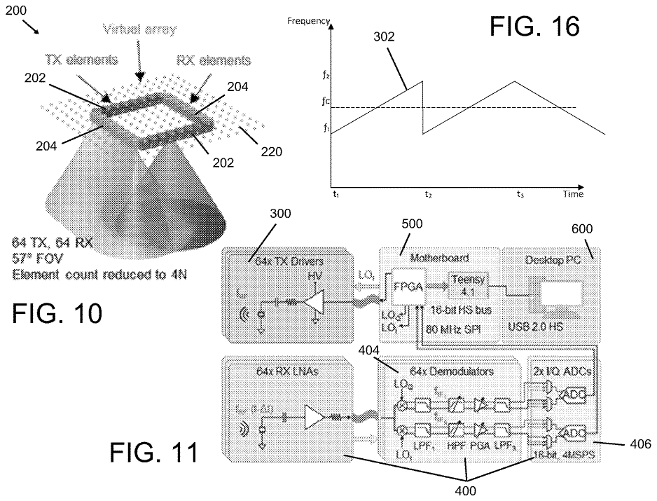

The transmit elements 202 and the receive elements 204 can be arranged on a surface of the substrate 206 around a portion or a perimeter region of the substrate 206 (e.g., as shown in FIG. 1) or along some or all sides of a polygonal perimeter region. The polygonal perimeter region can be, or define a square (e.g., as illustrated in FIGS. 3 and 10), a rectangle, an octagon, or another polygon. The polygonal perimeter region can have one or more pairs of opposing sides. The polygonal perimeter region can have an even number of sides, or can have an odd number of sides in some embodiments. In at least one embodiment, the polygonal perimeter region can be rectangular with two pairs of opposing sides. For example, the transmit elements 202 can be arranged along a first subset of sides of the polygonal perimeter region, such as one pair of opposing sides, and/or the receive elements 204 can be arranged along a second subset of sides of the polygonal perimeter region, such as another pair of opposing sides. The first subset of sides and the second subset of sides can be mutually exclusive, such that no side of the polygonal perimeter region contains both transmit and receive elements 202, 204. In at least one embodiment, the transmit elements 202 can arranged orthogonally to the receive elements 204. In at least one embodiment, the transmit elements 202 and the receive elements 204 can be arranged along any number of opposing sides, such as where the polygonal perimeter region is a square, a rectangle, or an octagon.

As shown in the exemplary embodiment of FIG. 3, a plurality of transmit elements 202 can be arranged on a substrate 206 along a first side of a polygonal perimeter region to form a first subarray 212, a plurality of receive elements 204 can be arranged on the substrate 206 along a second side of the polygonal perimeter region to form a second subarray 214, another plurality of transmit elements 202 can be arranged on the substrate 206 along a third side of a polygonal perimeter region to form a third subarray 216, another plurality of receive elements 204 can be arranged on the substrate 206 along a fourth side of the polygonal perimeter region to form a fourth subarray 218, or any combination thereof. In at least one embodiment, the first subarray 212 and/or the third subarray 216 can each contain 32 transmit elements 202. Similarly, in at least one embodiment, the second subarray 214 and/or the fourth subarray 218 can each contain 32 receive elements 204. While each subarray 212, 214, 216, 218, or side, can contain the same number of elements, 202, 204, any or all of the subarrays 212, 214, 216, 218, or sides, can contain different numbers of elements 202, 204. The first side and the third side can be opposing and/or the second side and the fourth side can be opposing, such as where the polygonal perimeter region is rectangular. Similarly, the first subarray 212 and the third subarray 216 can be opposing and/or the second subarray 214 and the fourth subarray 218 can be opposing. In at least one embodiment, no side of the polygonal perimeter region contains both transmit elements 202 and receive elements 204.

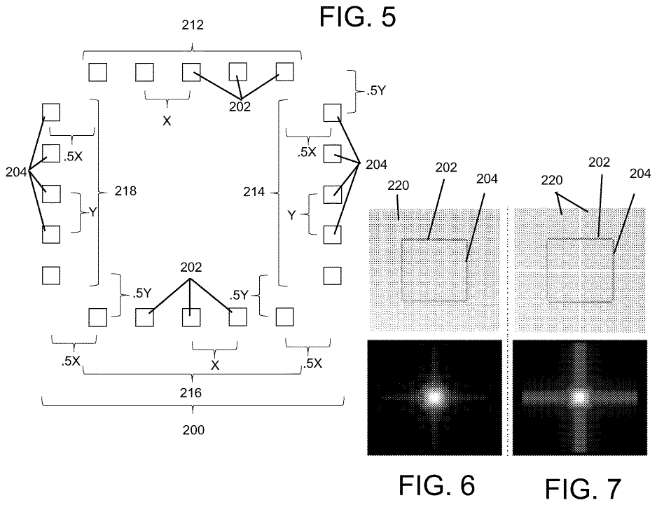

As shown in the exemplary embodiment of FIG. 5, the elements 202, 204 can be spaced at various pitches and/or the subarrays 212, 214, 216, 218 can be spaced or offset from each other, such as by multiples, fractions, or some other function of one or more of the pitches. For example, the transmit elements 202 in the first subarray 212 and/or the transmit elements 202 in the third subarray 216 can be spaced at a first pitch X. The receive elements 204 in the second subarray 214 and/or the receive elements 204 in the fourth subarray 218 can be spaced at a second pitch Y. The first pitch X and the second pitch Y can be the same or different. The first subarray 212 and/or the third subarray 216 can be offset from the second subarray 214 and/or the fourth subarray 218 by a first function of the first pitch X and/or the second pitch Y. Similarly, the second subarray 214 and/or the fourth subarray 218 can be offset from the first subarray 212 and/or the third subarray 216 by a second function of the first pitch X and/or the second pitch Y. The first function and the second function can be the same or different. In at least one embodiment, the first subarray 212 and/or the third subarray 216 can be offset from the second subarray 214 and/or fourth subarray 218 by approximately one half of the second pitch Y. The second subarray 214 and/or the fourth subarray 218 can be offset from the first subarray 212 and/or third subarray 216 by approximately one half of the first pitch X. In an exemplary embodiment, the first subarray 212 and/or the third subarray 216 can be spaced at a pitch of 1.0 and/or be offset from the second subarray 214 and/or fourth subarray 218 by approximately 0.5 and the second subarray 214 and/or the fourth subarray 218 can be spaced at a pitch of 1.5 and/or be offset from the first subarray 212 and/or third subarray 216 by approximately 0.75. While the elements 202, 204 in each subarray 212, 214, 216, 218 can be spaced at the same pitch X, Y, any of the elements 202, 204 in any or all of the subarrays 212, 214, 216, 218 can be spaced at different pitches. In at least one embodiment, the pitches can vary between 0.3 millimeters and 0.5 millimeters and/or be dependent upon the bandwidth and/or center frequency of the excitation signal 302. For example, the pitches can be a function of a wavelength of the center frequency or other frequency of the excitation signal 302.

The transmit elements 202 and/or the receive elements 204 can be generally linearly aligned in the various subarrays, 212, 214, 216, 218. In at least one embodiment, not all elements 202, 204 need to be perfectly aligned in the various subarrays 212, 214, 216, 218. For example, the transmit elements 202a at each end of the first subarray 212 can be disposed further from the third subarray 216 than the other transmit elements 202 in the first subarray 212, the receive elements 204a at each end of the second subarray 214 can be disposed further from the fourth subarray 218 than the other receive elements 204 in the second subarray 214, the transmit elements 202a at each end of the third subarray 216 can be disposed further from the first subarray 212 than the other transmit elements 202 in the third subarray 216, the receive elements 204a at each end of the fourth subarray 218 can be disposed further from the second subarray 214 than the other receive elements 204 in the fourth subarray 218, or any combination thereof. In at least one embodiment, this spacing can be according to a function of the first pitch X and/or the second pitch Y, such as discussed above. For example, as shown in FIG. 8, the transmit elements 202a at each end of the first subarray 212 can be disposed 0.5 Y (or half the pitch of the receive elements 204 in the second subarray 214 and/or the fourth subarray 218) further from the third subarray 216 than the other transmit elements 202 in the first subarray 212.

This spacing and/or offset, between the elements 202, 204 and/or the subarrays 212, 214, 216, 218 can be used in the alternative, can be added, can be subtracted can be related by some other function, or any combination thereof. For example, in at least one embodiment, all elements 202, 204 in each subarray 212, 214, 216, 218 can be linearly aligned within the subarrays 212, 214, 216, 218, with the subarrays 212, 214, 216, 218 themselves offset from one another as described herein. In other embodiments, the subarrays 212, 214, 216, 218 themselves need not be offset. Rather, just the elements 202a, 204a at each end of the subarrays 212, 214, 216, 218 can be offset. In at least one embodiment, the first subarray 212 can be generally offset from the second subarray 214 and/or fourth subarray 218 by approximately 0.5 Y, with the transmit elements 202a at each end of the first subarray 212 disposed a further 0.5 Y, such that some of the transmit elements 202 of the first subarray 212 are offset by approximately 0.5 Y and some are offset by approximately 1.0 Y. The other elements 202, 204 in the other subarrays 214, 216, 218 can be offset in the same manner, to the same degree, or differently. The subarrays 212, 214, 216, 218 and/or the elements 202, 204 therein can be offset according to a function of their own pitch X, Y, with or without a function of the pitch X, Y of the other subarrays 212, 214, 216, 218. For example, the first subarray 212 can be offset from the second subarray 214 and/or fourth subarray 218 by a function of X, the pitch of the first subarray 212 with or without Y, the pitch of the second subarray 214.

The transmit circuit 300, the receive circuit 400, the processor 500, or any combination thereof can be embodied in a single integrated circuit 110, such as an application-specific integrated circuit (ASIC). In at least one embodiment, as shown in FIG. 1, the transmit circuit 300 and the receive circuit 400, can be embodied in a single integrated circuit 110 and coupled to the processor 500 via one or more cables 112 and/or one or more wireless connections.

As shown in FIG. 3, the transducer array 200 and/or any or all of the subarrays 212, 214, 216, 218 can fully or partially surround the integrated circuit 110. Electrical connections between the integrated circuit 110 and the transducer array 200 can extend radially outwards from the integrated circuit 110.

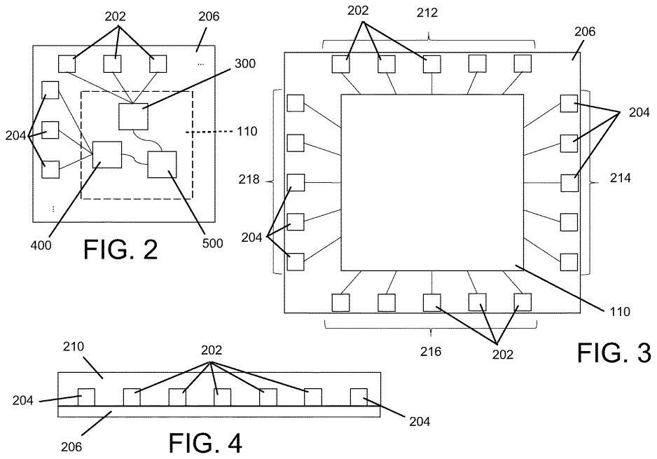

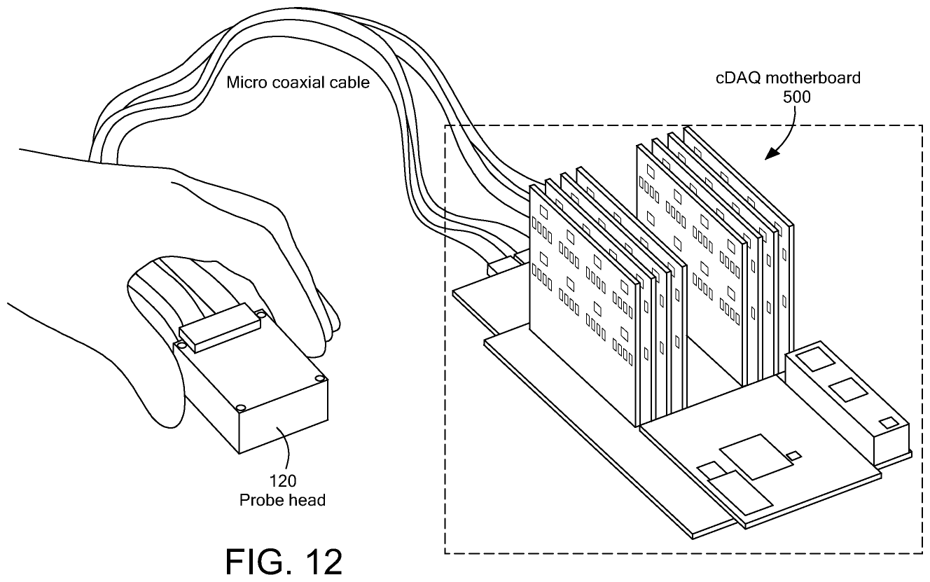

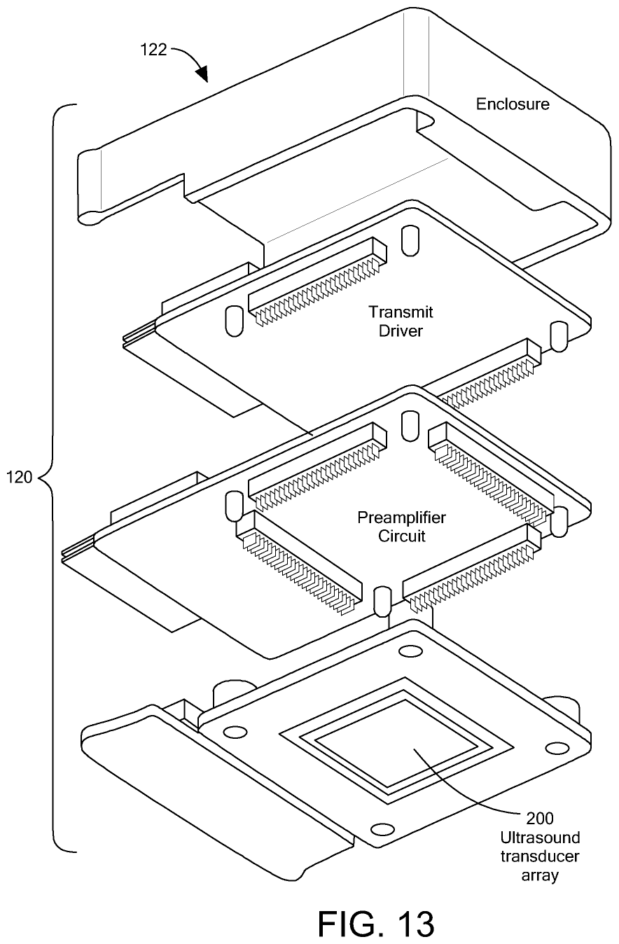

As shown in FIGS. 12 and 13, the transducer array 200, the transmit circuit 300, the receive circuit 400, or any combination thereof can be combined into a sensing probe 120. The sensing probe 120 can include an enclosure 122, which can house a transducer array (such as transducer array 200 described above in FIGS. 1, 5, 8, 10 and 13), the transmit elements (such as transmit elements 202 described above in FIGS. 1, 5, 8, 10 and 13), the receive elements (such as receive elements 204 described above in FIGS. 1, 5, 8, 10 and 13), the substrate (such as substrate 206 described above in FIGS. 1, 2 and 4), the conductive backing layer (such as conductive backing layer 210 described above in FIG. 4), the transmit circuit (such as transmit circuit 300 described above in FIGS. 1 and 11 and 4), the receive circuit (such as transmit circuit 400 described above in FIGS. 1 and 11), or any combination thereof.

The receive circuit 400 can provide a frequency down conversion of the analog signals 402 and/or digitize the analog signals 402. The receive circuit 400 can include one or more delta-sigma analog-to-digital converters that can provide both the frequency down conversion of the analog signals 402 and digitize the analog signals 402. The delta-sigma analog-to-digital converter can sample the analog signals 402 from the receive elements 204, such as at a sampling rate based at least in part on the swept-frequency and/or the center frequency. For example, a sampling gate of the delta-sigma analog-to-digital converter can switch based at least in part on the swept-frequency and/or the center frequency. The sampling rate can be a multiple of the swept-frequency or the center frequency. The digital signals from the delta-sigma analog-to-digital converter can be provided at a much lower rate, such as half the swept-frequency or the center frequency. In some embodiments, the digital signals from the delta-sigma analog-to-digital converter can be provided at one or more orders of magnitude less than swept-frequency or the center frequency. The sampling rate can vary over time, such as following the swept-frequency, or be constant, such as based on the center frequency.

The receive circuit 400 can include one or more analog frequency downconverters 404 and/or one or more analog-to-digital converters 406. The analog frequency downconverter 402 can produce one or more intermediate-frequency signals 408 for each of the receive elements, such as by mixing the analog signal 402 from respective receive elements with a reference signal and/or applying one or more filters. The reference signal can be based at least in part on the swept-frequency and/or the center frequency of the excitation signal 302. The intermediate-frequency signal 408 for each of the receive elements 204 can include an echo time delay, which can be caused by a depth of the object within the medium from which the acoustic energy is reflected or otherwise received. The echo time delay can be encoded as a proportional frequency, with or without an offset. The analog-to-digital converter 406 can produce the digital signal for each of the receive elements 204, such as by sampling the intermediate-frequency signal 408 for each of the receive elements 204 at a sampling rate. The sampling rate can be less than the swept-frequency and/or the center frequency of the excitation signal 302. For example, the sampling rate can be less than half the center frequency or an order of magnitude (or more) less than the center frequency.

The receive circuit 400 can include one analog frequency downconverter 404 and/or one analog-to-digital converter 406 for each of the receive elements 204. In at least one embodiment, the receive circuit 400 can include one analog frequency downconverter 404 for each of the receive elements 204 and fewer analog-to-digital converters 406. For example, the receive circuit 400 can include one or more multiplexers (MUX) to selectively feed the intermediate-frequency signal 408 for each of the receive elements 204 to one or more analog-to-digital converters 406. In at least one embodiment, the receive circuit 400 can include two MUXs, with one MUX feeding the intermediate-frequency signal 408 for each of the receive elements 204 in the second subarray 214, or the second side, to a first analog-to-digital converter 406 and another MUX feeding the intermediate-frequency signal 408 for each of the receive elements 204 in the fourth subarray 218, or the fourth side, to a second analog-to-digital converter 406. The MUXs can effectively provide two or more channels for converting the analog signals 402 and/or the intermediate-frequency signals 408 into the digital signals. While the analog signals 402 and/or the intermediate-frequency signals 408 can be channelized by subarray or side, they can be channelized according to other schemes, such as an alternating pattern, which can ensure simultaneous processing of the analog signals 402 and/or the intermediate-frequency signals 408 from receive elements 204 that are similarly and/or opposingly positioned. In exemplary embodiments, the MUXs, if used, can be placed and operate anywhere between the receive elements 204 and the processor 500. For example, the MUXs can selectively feed the analog signals 402 received from the receive elements 204 to one or more analog frequency downconverters 404, such as using MUXs with a sufficiently operating high speed which can be based on the swept-frequency and/or the center frequency, or selectively feed the digital signals from the analog-to-digital converters 406 to the processor 500, such as where there is an analog-to-digital converter 406 for each receive element 204.

The processor 500 can display the volumetric image, such as on a display (not shown), and/or provide the volumetric image to an external computer 600 for display and/or further analysis. The processor 500 can account for any time offsets, such as due to non-simultaneous processing of the analog signals 402 and/or the intermediate-frequency signals 408, which can be experienced with the multiplexed channel scheme discussed above. The processor 500 can control the MUXs and/or receive information from the receive circuit 400, such as the MUXs and/or analog-to-digital converters 406 identifying which digital signal to associate with which receive element 204 and/or position thereof. In at least one embodiment, the processor 500 can receive and/or process the digital for each of the receive elements 204 simultaneously, such as where each of the receive elements 204 has their own delta-sigma analog-to-digital converter, analog frequency downconverter 404, analog-to-digital converter 406, or any combination thereof. In some embodiments, the processor 500 can include the external computer 600, the external computer 600 can include the processor 500, the processor 500 and the external computer 600 can operate together, sharing functions, or any combination thereof. For example, the processor 500 can monitor and/or control the transmit circuit 300 and/or the receive circuit 400 and provide the digital signals to the external computer 600, which can construct the image from the digital signals using its own processor.

As shown in FIG. 14, the excitation signal 302 can be a constant power chirp across a predetermined frequency band, such as 0.5-7.0 Megahertz, over a predetermined duration, such as 7 milliseconds or 10 milliseconds. In at least one embodiment, the predetermined duration can be function of the frequency of the excitation signal 302, such as 100 or more full cycles of the excitation signal 302 or the center frequency. After the duration, the excitation signal 302 can repeat the same frequency sweep. As shown in FIG. 16, the excitation signal 302 can sweep up, such as from 0.5 Megahertz to 7.0 Megahertz, and/or down, such as from 7.0 Megahertz to 0.5 Megahertz, over the duration. In at least one embodiment, the excitation signal 302 can sweep up over a predetermined duration and then back down over the same or different predetermined duration, periodically or continuously. Sawtooth and triangular waveform shapes are illustrated in FIG. 16. However, it should be appreciated the waveform may be provided having any continuous periodic shape including but not limited to a sawtooth shape, a triangular shape, a sinusoidal shape (e.g. sin or arcsin), a substantially square wave shape, a substantially rectangular wave shape or any shape or combination of shapes; the waveform may also be provided having a non-periodic shape). After reading the disclosure provided herein, one or ordinary skill in the art will appreciate how to select a waveform shape to suit the needs of a particular application. In at least one embodiment, the analog signal 402 can be received from the receive elements 204 during the predetermined duration, periodically, continuously, while the transmit elements 202 are emitting acoustic energy, or any combination thereof. The analog signal 402 received from the receive elements 204 can include a pulse-echo frequency response of the receive element 204. For example, receiving the acoustic energy from the medium can be delayed from transmitting the acoustic energy from the medium, which can indicate a depth within the medium. In at least one embodiment, that and/or other delays can be encoded in the analog signal 402, such as by a proportional frequency.

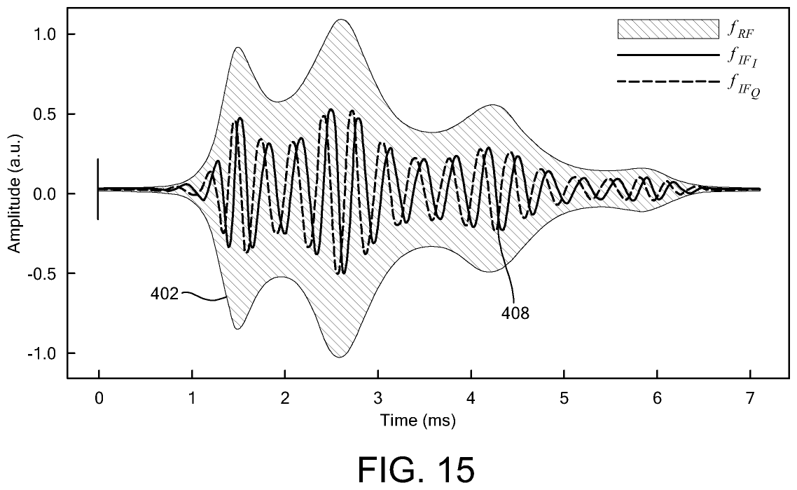

As shown in FIG. 15, the analog signal 402 can appear as a solid envelope due to the high frequency content, which can be based on the swept-frequency of the excitation signal 302. The intermediate-frequency signal 408 can contain the same information as the analog signal 402 at a much lower frequency, which can allow the analog-to-digital converters 406 to operate at a much lower frequency, be cheaper, consume less power, or any combination thereof.

In at least one embodiment, the array 200 can be a box-shaped array with separate and orthogonal transmit and receive subarrays 212, 214, 216, 218. While the array 200 can take other forms and/or shapes without departing from the scope of the disclosed inventions described herein, the box-shaped array can consume a smaller physical footprint for a given virtual aperture and/or provide other advantages over other forms and/or shapes tested. The convolution of the subarrays 212, 214, 216, 218 can define the virtual aperture, the size and distribution of which can determine the imaging resolution. The transducer array 200 can take the form of a convolutional optimally distributed array (CODA), which can provide the virtual aperture with no repeated samples and no gaps. The CODA 200 can achieve the sharpest far field image resolution with minimal image artifacts. The small transmit and receive elements 202, 204 can have a wide angle of sensitivity, allowing imaging well outside the footprint of the transducer array, or CODA, 200. In at least one embodiment, the CODA 200 can have 128 elements, such as 64 transmit elements 202 and 64 receive elements 204, and match the aperture size of a 1024 element full matrix array, achieving an 8× reduction in total elements and a 16× reduction in RX elements. For example, the CODA 200 disclosed herein can achieve the same combined wide angle imaging, such as a 57 degree imaging cone, and far field resolution as a full matrix array with O(N²) elements, with only O(4N) elements, simplifying fabrication and making high resolution volumetric imaging more practical. In some embodiments, the chirped data acquisition (cDAQ) can lower the voltage and/or sampling rates by more than an order of magnitude, while reducing the per-channel power consumption by approximately a factor of two compared to previous techniques. In at least one embodiment, the entire ultrasound imaging system 100 including the transducer array 200, cables 112, and data acquisition electronics 300, 400, can weigh as little as 1.15 lbs. The ultrasound imaging system 100 according to the disclosure can enable high performance, real time 4D ultrasound in a wearable device and/or otherwise remove key and persistent problems for such medical devices.

An exemplary CODA 200 can achieve a desired imaging resolution with minimal sidelobes or artifacts, while using a reduced number of transmit and/or receive elements 202, 204. Reducing the number of transmit and/or receive elements 202, 204 can reduce the resources needed to operate the transducer array 200, such as electronics, computing power, electrical power consumption, or any combination thereof. For example, the transmit and/or receive elements 202, 204 of the CODA 200 can be arranged to achieve a unity-valued non-redundant virtual array 220, which can be defined as the convolution of the elements 202, 204 in the various subarrays 212, 214, 216, 218. The non-redundancy in the virtual array 220 can minimize the electronics needed to interface with and operate the transducer array (or CODA) 200 and/or can minimize the computing power needed to construct the image from the information provided by the transducer array 200.

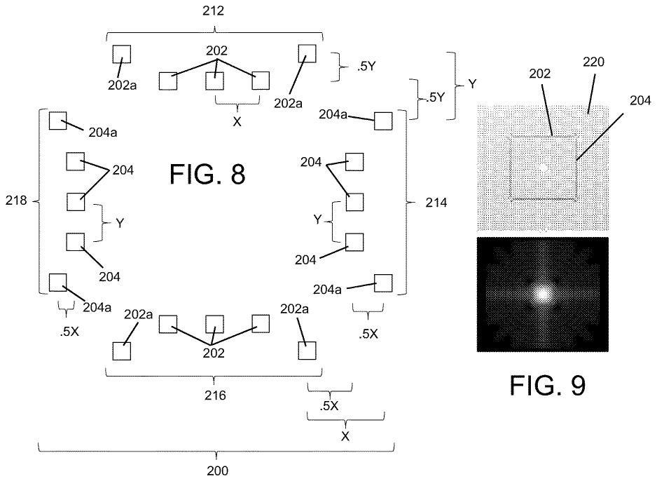

In at least one embodiment, the square version of the transducer array 200 shown in FIG. 6 provides high imaging resolution, which can be equal in both axial directions (x and y), using a low number of elements 202, 204. In some embodiments, the transmit elements 202 and the receive elements 204 can be linearly arranged with an offset between the subarrays 212, 214, 216, 218, such as shown in FIGS. 5 and 6. As shown in FIG. 7, as the offset between the subarrays 212, 214, 216, 218 increases, one or more gaps can begin to appear in the virtual array 220, which can degrade the sidelobe performance, cause artifacts, cause other performance issues, or any combination thereof. In other exemplary embodiments, the end elements 202a, 204a of each subarray 212, 214, 216, 218 can be offset in addition to or as an alternative to an offset between the subarrays 212, 214, 216, 218, such as shown in FIGS. 8 and 9, which can provide a more uniform virtual array 220, reduce cross-talk between the transmit elements 202 and the receive elements 204, improve the sidelobe performance, reduce artifacts, or any combination thereof.

It can be seen that, in each of FIGS. 6, 7, and 9, an arrangement of the transmit elements 202, the receive elements 204, and the virtual array 220 are shown above a corresponding point spread function (PSF), which characterizes the imaging resolution and sidelobe/artifact behavior of the virtual array 220. The embodiment of FIG. 6 shows the weakest sidelobes, followed by the embodiment of FIG. 9, then followed by the embodiment of FIG. 7. In at least one embodiment, an optimal spacing can be based on a spatial Nyquist condition, such as spacing the transmit and/or receive elements 202, 204 by ½ lambda at the frequency of the excitation signal 302, which can give a larger field of view.

The cDAQ ultrasound architecture according to the disclosure can use compressive sensing to boost the signal to noise ratio (SNR) at low system voltages while simultaneously realizing a 2 orders-of-magnitude reduction in sampling rate. The cDAQ, as disclosed herein, can perform the signal averaging operation in analog by down converting the analog signals 302 at a high received frequency, passively filtering, and sampling at a reduced frequency or rate, which can avoid energy-intensive oversampling and digital decimation altogether. As a result, the CODA 200 can reduce the number of power consuming receive channels by 16× and/or the cDAQ architecture can realize a further multiplicative reduction in per-channel power consumption of 1.85×, which can result in a 29.6× power reduction compared to a full matrix array with similar performance.

In at least one embodiment, the cDAQ architecture can use a swept-frequency continuous wave function:

$${f}_{chirp} ( t ) = {e}^{2 π i ( {ϕ}_{i} + {f}_{0} t + \frac{1 df}{2 dt} {t}^{2} )}$$

defined by the initial phase angle (φᵢ), the initial frequency (f₀), and the chirp ramp rate (df/dt). The complex-valued chirp can be digitally synthesized and thresholded on a field-programmable gate array (FPGA) using a numerically controlled oscillator (NCO), producing square wave in-phase:

LO_{I}=sgn(R(f_{chirp}))

and quadrature-phase:

LO_{Q}=sgn(J(f_{chirp})

copies of the signal. The LO_{I} signal can be power boosted by half-bridge transmit drivers to produce the excitation signal 302. The transmit elements 202 can then radiate the acoustic energy as diverging waves, which scatter and reflect from impedance discontinuities as they travel through the medium. The reflected and time delayed echo

f_{RF}(t−Δt)

can be captured by the receive elements 204 which can provide the analog signals 402 to the receive circuit 400, where they can be amplified by low noise amplifiers (LNA), demodulated by analog multiplication with the LO_{I} and LO_{Q} signals, low pass filtered (LPF), or any combination thereof to produce the intermediate-frequency signal 408

$${f}_{IF} ( t ) = {e}^{2 π i ( - {f}_{0} Δ t + \frac{1 df}{2 dt} Δ {t}^{2} - \frac{df}{dt} t Δ t )}$$

which can include translating the echo time delay (Δt) into a proportional frequency (df/dt Δt), which can be determined by a chirp ramp rate, such as a rate of change of the excitation signal 302. Small time delays can be converted to low intermediate frequencies (IF) denoted f_{IF}, such that a programmable high pass filter (HPF) and/or a programmable gain amplifier (PGA) can be used to suppress internal array reverberations and/or otherwise optimize the overall dynamic range. An analog to digital converter 406 can sample the intermediate-frequency signal 408 (f_{IF}) and send the digitized data to the processor 500.

In at least one embodiment, the array 200 (with 64 transmit elements 202 and 64 receive elements 204), receive low noise amplifiers (LNAs), and the transmit circuit 300 can be stacked vertically and/or otherwise be incorporated into a sensing probe 120, such as to minimize capacitive loading of the receive elements 204. The analog frequency downconverters 404 and the analog-to-digital converters 406 can be integrated or otherwise co-located with the processor 500, such as a Teensy 4.1 microcontroller. In an exemplary embodiment, a pair of I/Q sampling, 4 MHz analog-to-digital converters 406 can scan all 64 receive elements 204, with each receive element 204 being sampled at a sampling rate of just 124 kHz, which is much lower than the swept-frequency and/or the center frequency of the excitation signal 302, which can be 0.5-7.0 Megahertz and 3.75 Megahertz, respectively, in an exemplary embodiment. Due to the low sampling rate and reduced frequencies, the power consumption can be reduced to just 1.50 mW/(channel·MHz), such that the ultrasound imaging system 100 can be 1.85×more efficient compared to previous integrated circuit ultrasound chips, at similar or better image resolution.

In at least one embodiment, the ultrasound imaging system 100 according to the disclosure can transmit and receive the acoustic energy simultaneously, on-demand, periodically, continuously, of any combination thereof, such as utilizing the cDAQ architecture disclosed herein. In some instances, crosstalk between transmit elements 202 and receive elements 204 can impact a maximum transmit voltage, or power, without saturating the low noise amplifiers (LNAs). Corner elements 202, 204 of the array 200, or those elements 202, 204 at the ends of the subarrays 212, 214, 216, 218, can be especially susceptible to this issue. In at least one embodiment, the elements 202, 204 at the ends of the subarrays 212, 214, 216, 218 can be offset further from other elements 202, 204 in the same subarrays 212, 214, 216, 218 and/or a portion 208 of the substrate 206 can be removed. For example, as discussed herein, the portion 208 of the substrate 206 can be removed through laser etching or micro-machining, which can also be used to form the elements 202, 204 themselves.

In illustrative implementations of the concepts described herein, one or more computers (e.g., integrated circuits, microcontrollers, controllers, microprocessors, processors, field-programmable-gate arrays, personal computers, onboard computers, remote computers, servers, network hosts, or client computers) may be programmed and specially adapted: (1) to perform any computation, calculation, program or algorithm described or implied above; (2) to receive signals indicative of human input; (3) to output signals for controlling transducers for outputting information in human perceivable format; (4) to process data, to perform computations, to execute any algorithm or software, and (5) to control the read or write of data to and from memory devices. The one or more computers may be connected to each other or to other components in the system either: (a) wirelessly, (b) by wired or fiber optic connection, or (c) by any combination of wired, fiber optic or wireless connections.

In illustrative implementations of the concepts described herein, one or more computers may be programmed to perform any and all computations, calculations, programs and algorithms described or implied above, and any and all functions described in the immediately preceding paragraph. Likewise, in illustrative implementations of the concepts described herein, one or more non-transitory, machine-accessible media may have instructions encoded thereon for one or more computers to perform any and all computations, calculations, programs and algorithms described or implied above, and any and all functions described in the immediately preceding paragraph.

For example, in some cases: (a) a machine-accessible medium may have instructions encoded thereon that specify steps in a software program; and (b) the computer may access the instructions encoded on the machine-accessible medium, in order to determine steps to execute in the software program. In illustrative implementations, the machine-accessible medium may comprise a tangible non-transitory medium. In some cases, the machine-accessible medium may comprise (a) a memory unit or (b) an auxiliary memory storage device. For example, in some cases, while a program is executing, a control unit in a computer may fetch the next coded instruction from memory.

In some cases, one or more computers are programmed for communication over a network. For example, in some cases, one or more computers are programmed for network communication: (a) in accordance with the Internet Protocol Suite, or (b) in accordance with any other industry standard for communication, including any USB standard, ethernet standard (e.g., IEEE 802.3), token ring standard (e.g., IEEE 802.5), or wireless communication standard, including IEEE 802.11 (Wi-Fi®), IEEE 802.15 (Bluetooth®/Zigbee®), IEEE 802.16, IEEE 802.20, GSM (global system for mobile communications), UMTS (universal mobile telecommunication system), CDMA (code division multiple access, including IS-95, IS-2000, and WCDMA), LTE (long term evolution), or 5G (e.g., ITU IMT-2020).

As used herein, “including” means including without limitation. As used herein, the terms “a” and “an”, when modifying a noun, do not imply that only one of the noun exists. As used herein, unless the context clearly indicates otherwise, “or” means and/or. For example, A or B is true if A is true, or B is true, or both A and B are true. As used herein, “for example”, “for instance”, “e.g.”, and “such as” refer to non-limiting examples that are not exclusive examples. The word “consists” (and variants thereof) are to be give the same meaning as the word “comprises” or “includes” (or variants thereof).

The above description (including any attached drawings and figures) illustrate example implementations of the concepts described herein. However, the concepts described herein may be implemented in other ways. The methods and apparatus which are described above are merely illustrative applications of the principles of the described concepts. Numerous modifications may be made by those skilled in the art without departing from the scope of the invention. Also, the described concepts include without limitation each combination, sub-combination, and permutation of one or more of the abovementioned implementations, embodiments and features.

Various embodiments of the concepts, systems, devices, structures and techniques sought to be protected are described herein with reference to the related drawings. Alternative embodiments can be devised without departing from the scope of the concepts, systems, devices, structures and techniques described herein.

It is noted that various connections and positional relationships (e.g., over, below, adjacent, etc.) are set forth between elements in the description and in the drawings. These connections and/or positional relationships, unless specified otherwise, can be direct or indirect, and the described concepts, systems, devices, structures and techniques are not intended to be limiting in this respect. Accordingly, a coupling of entities can refer to either a direct or an indirect coupling, and a positional relationship between entities can be a direct or indirect positional relationship.

The following definitions and abbreviations are to be used for the interpretation of the specification. As used herein, the terms “comprises,” “comprising, “includes,” “including,” “has,” “having,” “contains” or “containing,” or any other variation thereof, are intended to cover a non-exclusive inclusion. For example, a composition, a mixture, process, method, article, or Apparatus that comprises a list of elements is not necessarily limited to only those elements but can include other elements not expressly listed or inherent to such composition, mixture, process, method, article, or apparatus.

The terms “one or more” and “more than one” are understood to include any integer number greater than or equal to one, i.e. one, two, three, four, etc. The terms “a plurality” are understood to include any integer number greater than or equal to two, i.e., two, three, four, five, etc.

The term “connection” can include an indirect “connection” and a direct “connection.”

References in the specification to “one embodiment, “an embodiment,” “an example embodiment,” etc., indicate that the embodiment described can include a particular feature, structure, or characteristic, but every embodiment can include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Additionally, the terms “for example” and “exemplary” are used herein to mean “serving as an example, instance, or illustration. Any embodiment or design described herein as “exemplary” is not necessarily to be construed as preferred or advantageous over other embodiments or designs.

For purposes of the description hereinafter, the terms “upper,” “lower,” “right,” “left,” “vertical,” “horizontal, “top,” “bottom,” and derivatives thereof shall relate to the described structures and methods, as oriented in the drawing figures.

The terms “overlying,” “atop,” “on top, “positioned on” or “positioned atop” mean that a first element, such as a first structure, is present on a second element, such as a second structure, where intervening elements such as an interface structure can be present between the first element and the second element.

The term “direct contact” means that a first element, such as a first structure, and a second element, such as a second structure, are connected without any intermediary elements.

Use of ordinal terms such as “first,” “second,” “third,” etc., in the specification to modify an element does not by itself connote any priority, precedence, or order of one element over another or the temporal order in which acts of a method are performed, but are used merely as labels to distinguish one element having a certain name from another element having a same name (but for use of the ordinal term) to distinguish the elements.

The terms “substantially,” “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments.

The term “substantially,” “approximately” and “about” may also be used to refer to values that are within ±20% of a comparative measure in some embodiments, within ±10% in some embodiments, within ±5% in some embodiments, and yet within ±2% in some embodiments. For example, a first direction that is “substantially” perpendicular to a second direction may refer to a first direction that is within ±20% of making a 90° angle with the second direction in some embodiments, within ±10% of making a 90° angle with the second direction in some embodiments, within ±5% of making a 90° angle with the second direction in some embodiments, and yet within ±2% of making a 90° angle with the second direction in some embodiments.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter.

Accordingly, although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

The inventions have been described in the context of preferred and other embodiments and not every embodiment of the inventions has been described. Obvious modifications and alterations to the described embodiments are available to those of ordinary skill in the art having the benefits of the present disclosure. The disclosed and undisclosed embodiments are not intended to limit or restrict the scope or applicability of the inventions conceived of by the Applicant, but rather, in conformity with the patent laws, Applicant intends to fully protect all such modifications and improvements that come within the scope or range of equivalents of the following claims.

What is claimed is:

1. An ultrasound imaging system comprising:
a two-dimensional transducer array comprising
a plurality of transmit elements disposed on a substrate; and
a plurality of receive elements disposed on the substrate;
wherein the transmit elements and the receive elements are arranged on the substrate to define a virtual aperture therebetween and wherein the receive elements are configured to receive acoustic energy from a medium while each transmit element is emitting acoustic energy into the medium;
a transmit circuit configured to drive the transmit elements with an excitation signal at a swept-frequency that varies over time across a predetermined bandwidth and has a center frequency;
a receive circuit configured to receive an analog signal from each of the receive elements and produce a digital signal for each of the receive elements; and
a processor configured to construct a volumetric image from the digital signals.

2. The ultrasound imaging system of claim 1, wherein:
a. each of the transmit elements and each of the receive elements occupy distinct, non-overlapping positions; and
b. the transmit elements and the receive elements are arranged so that the virtual aperture is substantially fully sampled and substantially uniformly weighted.

3. The ultrasound imaging system of claim 1, wherein the transmit elements are arranged along a first subset of sides of a polygonal perimeter region; wherein the receive elements are arranged along a second subset of sides of the polygonal perimeter region; and wherein no side of the polygonal perimeter region contains both transmit and receive elements.

4. The ultrasound imaging system of claim 1, wherein the transmit elements are arranged orthogonally to the receive elements.

5. The ultrasound imaging system of claim 1, wherein the transmit circuit and the receive circuit are embodied in a single integrated circuit; wherein the transducer array at least partially surrounds the integrated circuit; and wherein electrical connections between the integrated circuit and the transducer array extend radially outwards from the integrated circuit.

6. The ultrasound imaging system of claim 1, wherein the receive circuit comprises at least one delta-sigma analog-to-digital converter that samples the analog signals from each of the receive elements at a sampling rate based at least in part on the swept-frequency.

7. The ultrasound imaging system of claim 1, wherein the receive circuit comprises
at least one analog frequency downconverter configured to produce an intermediate-frequency signal for each of the receive elements by mixing the analog signal from the respective receive element with a reference signal that is based at least in part on the swept-frequency; and
at least one analog-to-digital converter configured to produce the digital signal for each of the receive elements by sampling the intermediate-frequency signal for each of the receive elements at a sampling rate that is less than half the center frequency.

8. The ultrasound imaging system of claim 7, wherein the intermediate-frequency signal for each of the receive elements includes an echo time delay encoded as a proportional frequency.

9. An ultrasound imaging system comprising:
a two-dimensional transducer array comprising
a first plurality of transmit elements arranged on a substrate along a first side of a polygonal perimeter region to form a first subarray;
a second plurality of receive elements arranged on the substrate along a second side of the polygonal perimeter region to form a second subarray;
a third plurality of transmit elements arranged on the substrate along a third side of a polygonal perimeter region to form a third subarray; and
a fourth plurality of receive elements arranged on the substrate along a fourth side of the polygonal perimeter region to form a fourth subarray;
wherein each transmit element is configured to emit acoustic energy into a medium in response to an excitation signal; and
wherein each receive element is configured to provide an analog signal in response to acoustic energy received from the medium while each transmit element is emitting the acoustic energy into the medium;
a transmit circuit configured to provide the excitation signal at a swept-frequency that varies over time across a predetermined bandwidth and has a center frequency;
a receive circuit configured to receive the analog signal from each of the receive elements and produce a digital signal for each of the receive elements; and
a processor configured to construct a volumetric image from the digital signals.

10. The ultrasound imaging system of claim 9, wherein no side of the polygonal perimeter region contains both transmit elements and receive elements.

11. The ultrasound imaging system of claim 9, wherein the polygonal perimeter region is rectangular; wherein the first side and the third side are opposing; and wherein the second side and the fourth side are opposing.

12. The ultrasound imaging system of claim 9, wherein the transmit elements in the first subarray and the transmit elements in the third subarray are spaced at a first pitch;
wherein the receive elements in the second subarray and the receive elements in the fourth subarray are spaced at a second pitch; wherein the first subarray and the third subarray are offset from the second subarray and fourth subarray by approximately one half of the second pitch; and wherein the second subarray and the fourth subarray are offset from the first subarray and third subarray by approximately one half of the first pitch.

13. The ultrasound imaging system of claim 9, wherein:
a. the transmit elements at each end of the first subarray are disposed further from the third subarray than the other transmit elements in the first subarray;
b. the receive elements at each end of the second subarray are disposed further from the fourth subarray than the other receive elements in the second subarray;
c. the transmit elements at each end of the third subarray are disposed further from the first subarray than the other transmit elements in the third subarray; and
d. the receive elements at each end of the fourth subarray are disposed further from the second subarray than the other receive elements in the fourth subarray.

14. The ultrasound imaging system of claim 9, wherein the substrate is thinner between adjacent subarrays.

15. The ultrasound imaging system of claim 9, further including a conductive backing layer;
and wherein the transmit elements and the receive elements are disposed between the substrate and the conductive backing layer.

16. The ultrasound imaging system of claim 9, wherein:
a. the transmit circuit and the receive circuit are embodied in a single integrated circuit;
b. the transducer array at least partially surrounds the integrated circuit; and
c. electrical connections between the integrated circuit and the transducer array extend radially outwards from the integrated circuit.

17. The ultrasound imaging system of claim 9, wherein the receive circuit comprises:
a. at least one delta-sigma analog-to-digital converter that samples the analog signals from each of the receive elements at a sampling rate based at least in part on the swept-frequency
b. at least one analog frequency downconverter configured to produce an intermediate-frequency signal for each of the receive elements by mixing the analog signal from each of the receive elements with a reference signal based at least in part on the swept-frequency;
c. at least one analog-to-digital converter configured to produce the digital signal for each of the receive elements by sampling the intermediate-frequency signal for each of the receive elements at a sampling rate that is less than half the center frequency; and
d, wherein the intermediate-frequency signal for each of the receive elements includes an echo time delay encoded as a proportional frequency.

18. An ultrasound transducer system comprising:
a plurality of transmit elements arranged along at least a first side of a polygonal perimeter region and configured to emit acoustic energy into a medium in response to an excitation signal; and
a plurality of receive transducer elements arranged along at least a second side of the polygonal perimeter region and configured to provide an analog signal in response to acoustic energy received from the medium;
wherein the first side and the second side are mutually exclusive;
wherein a two-dimensional spatial convolution of the transmit elements with the receive elements defines a fully sampled, unity-valued two-dimensional array having no unsampled spatial positions and no redundantly sampled spatial positions within its extent.

19. The ultrasound transducer system of claim 18, wherein:
a. the polygonal perimeter region is rectangular having the first side, the second side, a third side, and a fourth side;
b. the first side and the third side are opposing;
c. the second side and the fourth side are opposing;
d. the transmit elements are arranged along the first side and the third side; and
e. the receive elements are arranged along the second side and the fourth side.

20. The ultrasound transducer system of claim 19, wherein:
a. the transmit elements on the first side are spaced at a first pitch to form a first subarray;
b. the receive elements on the second side are spaced at a second pitch to form a second subarray;
c. the transmit elements on the third side are spaced at the first pitch to form a third subarray; and
d. the receive elements on the fourth side are spaced at the second pitch to form a fourth subarray.

21. The ultrasound transducer system of claim 20, wherein:
a. the first subarray and the third subarray are offset from the second subarray and fourth subarray by approximately one half of the second pitch; and
b. the second subarray and the fourth subarray are offset from the first subarray and third subarray by approximately one half of the first pitch.

22. The ultrasound transducer system of claim 21, wherein:
a. the transmit elements at each end of the first subarray are disposed further from the third subarray than the other transmit elements in the first subarray;
b. the receive elements at each end of the second subarray are disposed further from the fourth subarray than the other receive elements in the second subarray;
c. the transmit elements at each end of the third subarray are disposed further from the first subarray than the other transmit elements in the third subarray; and
d. the receive elements at each end of the fourth subarray are disposed further from the second subarray than the other receive elements in the fourth subarray.
